(19) United States
(12) Patent Application Publication
(10) Pub. No.: US 2012/0046571 A1
(43) Pub. Date: Feb. 23, 2012

(54) METHOD AND APPARATUS FOR DETECTING ETHANOL

(75) Inventors: Timo Varpula, Vtt (FI); Aarne Oja, Vtt (FI); Heikki Seppa, Vtt (FI); Mika Suhonen, Vtt (FI)

(73) Assignee: Teknologian tutkimuskeskus VTT, Espoo (FI)

(21) Appl. No.: 13/203,319

(22) PCT Filed: Feb. 23, 2010
(86) PCT No.: PCT/FI1200/050121

§ 371 Date: Sep. 13, 2011

(30) Foreign Application Priority Data
Feb. 26, 2009 (FI) 20095187

(51) Int. Cl. A61B 5/053

(52) U.S. Cl. 600/547

(57) ABSTRACT

The present publication discloses a method for detecting ethanol, in which method the ethanol content of a person is measured. According to the invention, the ethanol content is measured from the person's skin, using a capacitive measurement method.

The present invention relates to a method, according to the preamble of claim 1, for detecting ethanol.

The invention also relates to an apparatus for detecting ethanol.

At present, the detection of ethanol in the human body is mainly implemented by blood tests, or alternatively by using a breathalyser.

The detection of ethanol in the human body is also implemented by using a method based on a transdermal test. In this method, a sensor placed on the surface of the skin measures the amount of ethanol in a gaseous form. Ethanol travels through the skin partly in a gaseous form from the tissue fluids and along with perspiration. The commercial product SCRAM measures the ethanol content in a gaseous form from the surface of the skin electrochemically, using a method based on a fuel cell. In the WrisTAS prototype device, there is a platinum electrode, which oxidizes ethanol and the device measures the oxidation current, which is proportional to the amount of ethanol. Both SCRAM and WrisTAS are intended for the long-term monitoring of alcohol use. They are used in the USA, for instance, for the remote surveillance of alcohol use by prisoners. These devices are not suitable for the rapid measurement of alcohol content, for instance, in automobile immobilizer devices. In addition, research has shown that the content of ethanol in a gaseous form on the surface of the skin correlates with the blood alcohol content with a delay, which can reach a maximum of up to 120 min.

Ethanol content is also measured chemically from urine. However, the ethanol content of urine does not correlate very well with the ethanol content of blood, but varies strongly, for example, according to whether the bladder has been emptied or not prior to drinking alcohol. The ethanol content of blood has been regarded as the most important parameter, because blood ethanol directly affects brain operation and thus behaviour, reaction ability, and sensory operation. In practice, urine tests have been given up for the aforementioned reasons.

Blood tests are not suitable for daily use, such as in automobile immobilizer devices. There are on the market breathalysers, which are blown into. In these, alcohol is detected in the respiratory gas optically using a simple sensor based on infrared technology, or electrochemically using a sensor based on a fuel cell. There are also sensors based on metal oxides (for example, SnO₂) on the market, but their accuracy is less than that of infrared and electrochemical sensors. Present breathalysers are relatively reliable. The cheaper devices cost well under [symbol] 100, but more accurate devices cost several hundred euros. Alcoholmeters that require blowing are difficult to use, especially in vehicles. Disposal mouthpieces must be used with them and the manner of blowing has been observed to affect the measurement result. There is a great demand on the market for alcoholmeters that could be installed in a steering wheel or ignition switch and which could easily measure alcohol content without blowing.

The invention is intended to eliminate the defects of the state of the art described above and for this purpose create an entirely new type of method and apparatus for detecting ethanol.

The invention is based on measuring the impedance of the skin at a specific frequency range in detection.

More specifically, the method according to the invention is characterized by what is stated in the characterizing portion of claim 1.

For its part, the apparatus according to the invention is characterized by what is stated in the characterizing portion of claim 13.

Considerable advantages are gained with the aid of the invention.

The invention can be used to reduce traffic deaths. This is an important matter, as, in Europe for example, there are more than 40 000 traffic deaths annually. About 10 000 of these accidents are caused by drunken driving. In the USA about 15 000 and in Finland more than 100 people die each year in road-traffic accidents, in which a person under the influence of alcohol is involved. The number of injured and permanently disabled is an order of magnitude greater. The matter is thus concerns an important social, economic, health, and safety-related problem, for the solution of which the invention provides a simple, cheap, and effective tool.

The invention is particularly suitable for use in vehicles, such as road and rail vehicles, as well as vessels in water traffic and aircraft. The invention is also suitable for safety-critical workplaces, where it can be implemented combined with a fingerprint reader and an alcohol lock, which act as personal identification and a power or door lock. The device will probably be used first in professional traffic.

The invention has significant market potential. Worldwide, the production volume of new cars is fully 60 million annually. If it is assumed that a sensor system according to the invention (either as a purecapacitive alcohol meter or combined with a fingerprint reader and alcohol lock) is installed in 10% of them, we are talking of annual sales of millions of apparatuses.

According to one embodiment of the invention, personal identification can be combined with alcohol detection in the same device, and such a combination can also be used as a so-called alcohol lock, which can actively prevent the use of a vehicle by someone under the influence of drink.

The use of the device and method according to the invention is simple—contact of the skin with the sensor part of the device is all that is needed. The contact can be made using a finger, palm, or in principle any part of the skin. Disposable components such as blowing tubes are not required. In its entirety, that device is cheap and can be integrated in, for example, the steering wheel or gear lever of a car. A combined alcoholmeter and fingerprint reader is especially suitable for vehicular use: the sensor simultaneously identifies the user's fingerprint and measures the alcohol content—both using the same technology. An ignition key will then not be required at all and the device will also act as an alcohol lock. In a combined alcohol meter/fingerprint sensor, there is a common sensor element, reading electronics, and signal processing, which brings significant cost benefits. The manufacturing costs of the device are of the same order as those of capacitive fingerprint sensors. The alcoholmeter according to the invention measures the alcohol content of cell fluids in liquid form. This corresponds strongly with the blood alcohol content. There is no delay in the measurement, which is typical of transdermal sensors, such as SCRAM and WrisTAS measuring alcohol in gaseous form from the surface of the skin.

In the following, the invention is examined with the aid of examples and with reference to the accompanying drawings.

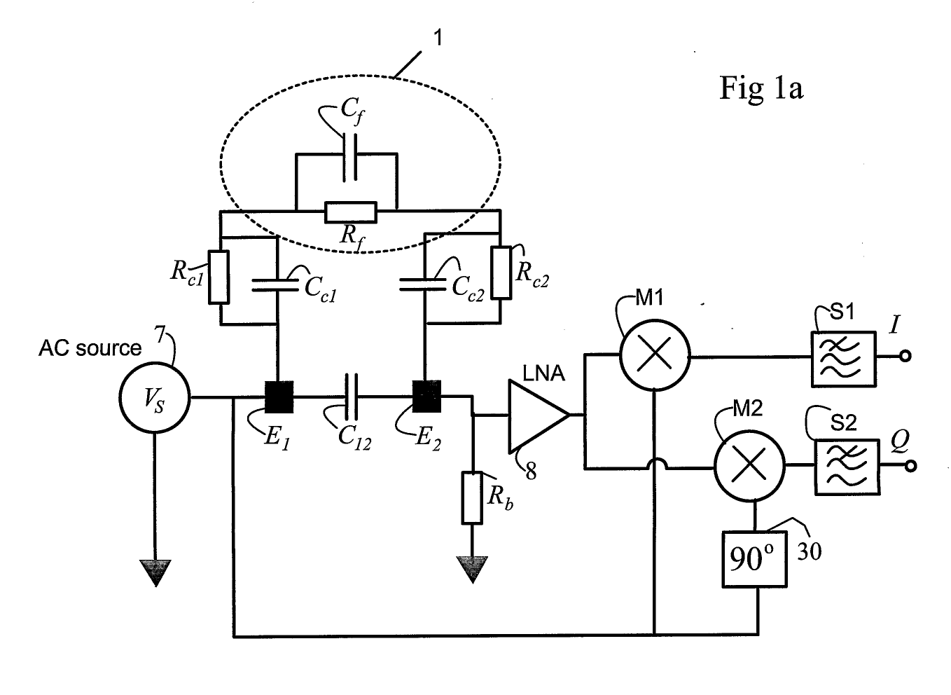

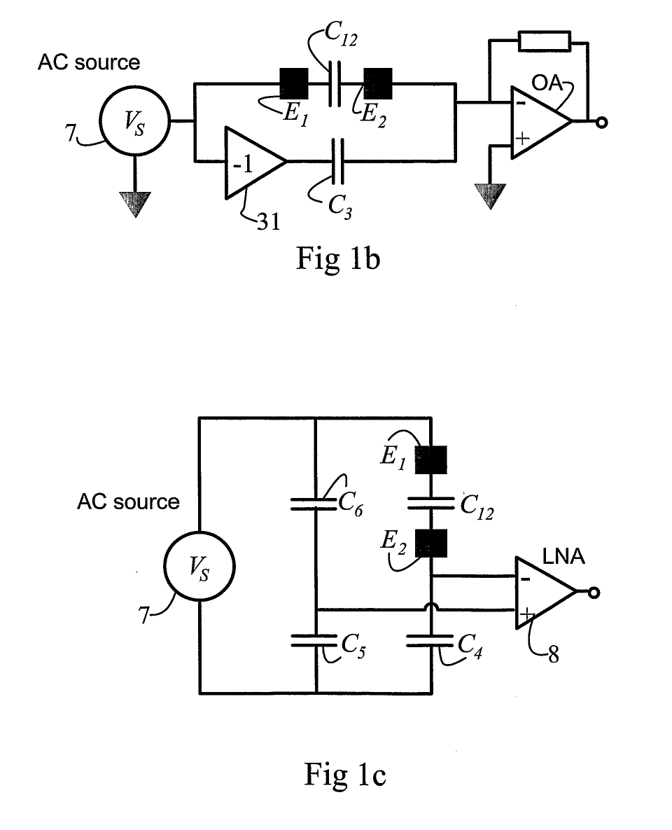

FIG. 1a shows schematically an equivalent circuit of connecting a finger or another measurement object to the measurement electrodes, as well as a measurement apparatus according to the invention.

FIGS. 1b and 1c show alternative measurement circuits according to the invention.

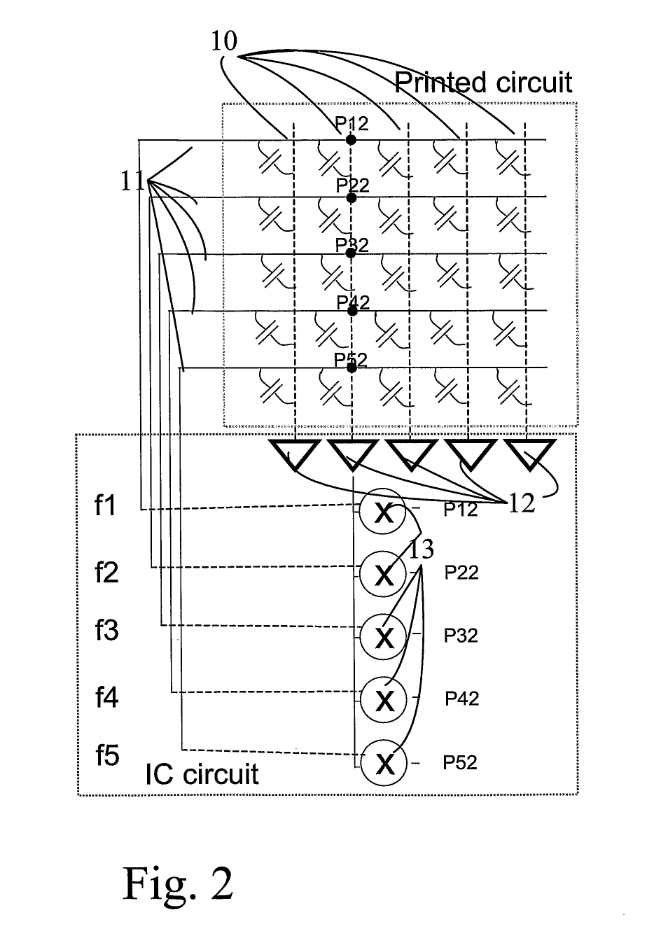

FIG. 2 shows schematically part of the measurement matrix according to the invention together with its ancillary connections.

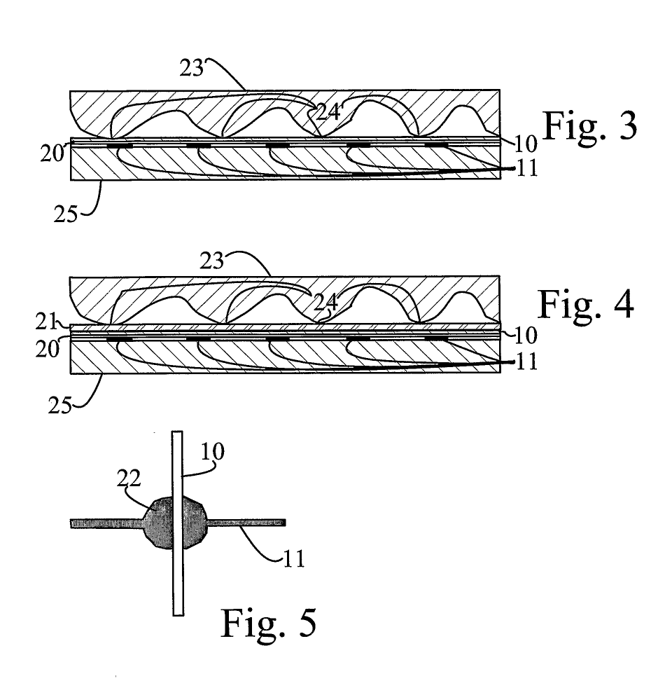

FIG. 3 shows a cross-sectional side view of one application according to the invention.

FIG. 4 show a cross-sectional side view of a second application according to the invention.

FIG. 5 shows a top view of one connection point of conductors.

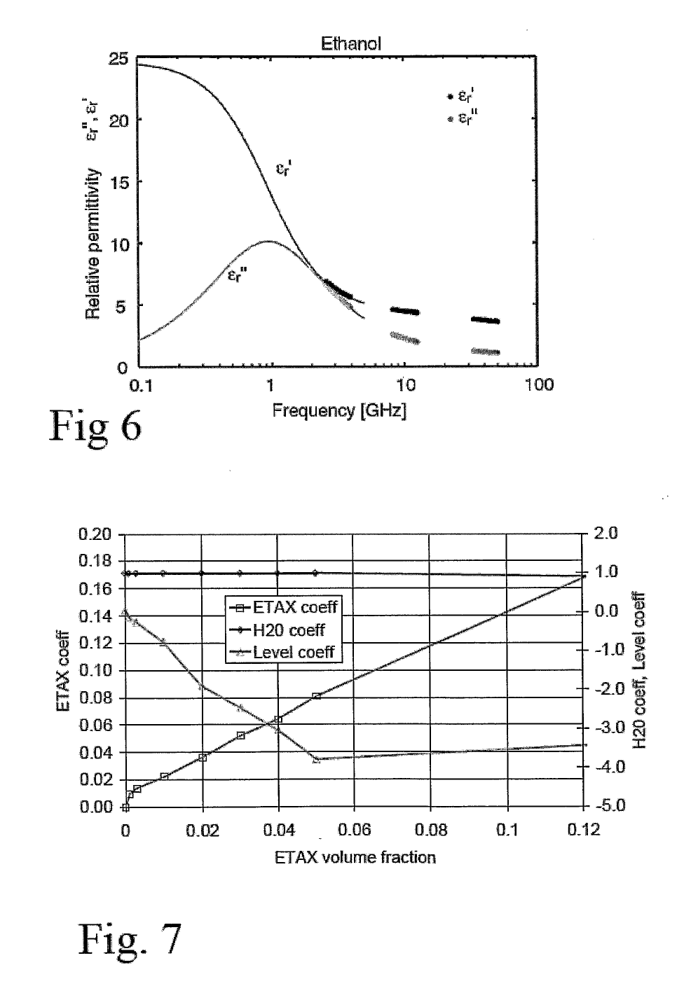

FIG. 6 shows graphically the electrical properties of an ethanol-water solution.

FIG. 7 shows graphically the application of a graph 1 to the measurement results of the permittivity of an ethanol and water solution, as a function of the volume fraction of ethanol.

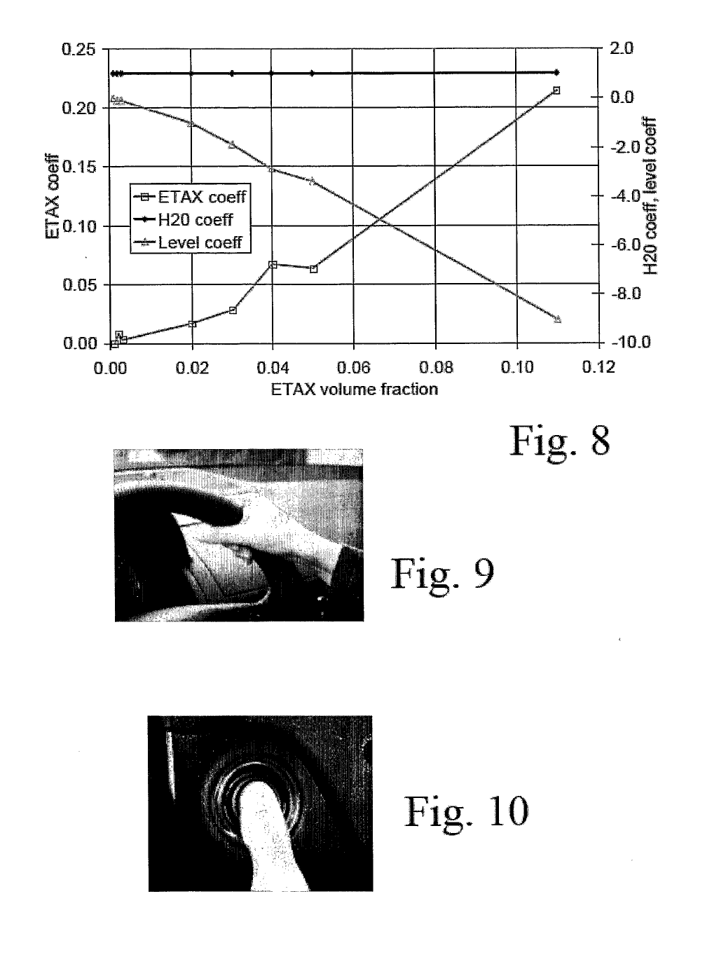

FIG. 8 shows graphically the application of a graph 1 to the measurement results of the permittivity of an ethanol and table-salt water solution, as a function of the volume fraction of ethanol.

FIG. 9 shows the use of a car steering wheel as a location for a measurement sensor according to the invention.

FIG. 10 shows the use of a separate measurement sensor located on the dashboard for both fingerprint and ethanol detection.

The following terminology is used in connection with the figures:
Equivalent circuit of skin 1
skin resistance R_{f}
skin capacitor C_{f}
1. coupling resistor R_{c1}
2. coupling resistor R_{c2}
1. coupling capacitor C_{c1}
2. coupling capacitor C_{c2}
voltage source 7
amplifier 8
1. electrode E₁
2. electrode E₂
parasitic capacitance C₁₂
vertical conductors 10
horizontal conductors 11
amplifiers 12
insulator layer/pyroelectric layer 20
pyroelectric layer 21
electrode extension 22
object (e.g., finger) being measured 23
contact points 24
substrate 25
phase inverter 30
inverter 31
measurement frequenceies f1-f5
measurement points P12-P52
first mixer M1
second mixer M2
first filter S1
second filter S2
operation amplifier OA
reference resistor R_{b}
reference capacitors C₃-C₆

According to the invention, the fingerprint is measured using the capacitive measurement principle. In the following, the invention is described in an example device environment.

According to the equivalent circuit of FIG. 1a, the electrodes E₁ and E₂ are located on a flat substrate. The electrodes E₁ and E₂ are connected to each other capacitively both in the substrate and through the power lines of the electrical field running above it. The connection occurring through the air changes when a dielectric or conductive material is placed on the substrate. The voltage between the electrodes can then be modelled in the manner depicted by the equivalent circuit and thus it is possible to measure the effect of the electrical components of the finger 1 between the electrodes E₁ and E₂.

In FIG. 1a, a measurement signal Vₛ is produced by the voltage source 7, from which the measurement signal is taken to a simple impedance bridge, which comprises the measured skin impedance 1 connected through the electrodes E₁ and E₂, and a reference resistor R_{b}. The impedance is connected from its centre to a sensitive amplifier 8. After the amplifier 8, the measurement current created and its phase are read in order to determine the skin impedance. The measurement signal is thus connected from the electrically conductive electrodes E₁ and E₂ either capacitively or directly to the skin, through the contact capacitors C_{c1} and C_{c2} and the contact resistors R_{c1} and R_{c2}. The impedance 1 of the skin being measured is depicted by the parallel connection of the resistor R_{f} and C_{f}. C₁₂ is the parasitic capacitance between the electrodes E₁ and E₂. By using the circuit of FIG. 1a to measure the impedance parameters R_{f} and C_{f} depicting the skin, in the frequency range 0.1-4 GHz, it is thus possible to determine both the ethanol content of the skin and the shape of the skin, with the aid of an electrode matrix.

FIG. 1a shows one way of implementing the impedance measurement. It is performed using two mixers M1 and M2. M1 detects the signal component in the same phase as the voltage V, produced by the voltage source 7, of the output of the amplifier LNA 8, and M2 detects the 90-degree phase-shifted component. The outputs of the mixers M1 and M2 are connected to filters S1 and S2. The output I of the filter S1 is proportional to the amplitude of the same-phase signal and the output Q of the filter S2 is the amplitude of the 90-degree phase-shifted signal.

FIG. 1b shows an example of an alternative way to implement the front end of the reading electronics of the sensor, based on a bridge circuit. The signal Vₛ of the voltage source 7 is connected through the electrodes E₁ and E₂ to an operation amplifier OA. The electrodes E₁ and E₂ are connected to the skin in accordance with FIG. 1a. The signal Vₛ of the voltage source 7 is also connected through an inverter to a capacitor C₃, which acts as a reference impedance. In turn it is connected to the amplifier OA. If the bridge is in equilibrium, i.e. the impedances in both branches of the bridge are of equal magnitude, the amplifier output will be zero. When the skin connects to the electrodes E₁ and E₂, there will be an alternating voltage in the output of the amplifier OA, the amplitude and phase of which will depend on the measured impedance of the skin.

FIG. 1c shows an example of an alternative way of implementing the front end of the reading electronics of the sensor, based on a second bridge circuit. The electrodes E₁ and E₂ connecting to the impedance of the skin are connected to a bridge, which is formed by the parasitic capacitance C₁₂ between the electrodes, and the reference capacitors C₄, C₅, and C₆. The voltage source Vₛ feeds current to the bridge. Assume that the bridge is in equilibrium before the skin connects to the electrodes E₁ and E₂; in other words, the output of the differential amplifier LNA is zero. When the skin connects to the electrodes E₁ and E₂, there will be an alternating voltage in the output of the amplifier LNA, the amplitude and phase of which will depend on the measured impedance of the skin.

Fingerprint modelling demands several image points. The actual electrodes could be patterned on a substrate like a circuit board, as long as the electrode conductors and the vias can be made at a sufficiently small resolution (50 micrometres). Using this technique, a problem arises in the electrical connection of the image pixels to the integrated circuit, which measures the impedance of each pixel. An entire fingerprint may consist of thousands of pixels. Making this many connections would require very highly developed and expensive connection technology.

According to the invention, the connection problem can be solved by using the multiplexing technique shown in FIG. 2. In FIG. 2, the presentation of FIG. 1 represents the equivalent circuit of one contact situation in the contact situation at the intersection of the conductors 10 and 11. According to the invention, the sensor structure of the position of an M×N image pixel is formed of an M×N conductor matrix, each of which M horizontal conductors 11 is connected to its own input signal and each of which N vertical conductors 10 is connected to its own current amplifier 12. The current amplifier 12 is able to position the current of its measured image pixel P12, P22, P32, P42, or P52, using either time or frequency-level multiplexing.

The horizontal conductors 11 and the vertical conductors 10 are connected to each other principally capacitively. Their intersections P12, P22, P32, P42, or P52 form a similar electrode pair to the electrode pair E₁ and E₂ of FIG. 1. The capacitive connection of conductors 10 and 11 is implemented by placing the horizontal 11 and vertical conductors 10 on different layers, separated by insulation. The upper electrodes can be either the horizontal 11 or the vertical conductors 10. Sensitivity can be increased by improving the geometry of the conductors 10 and 11. For example, the upper conductors can be insulated from the areas that are not at the intersections of the conductors. The intersections can also be widened according to FIG. 5, in order to improve the connection of particularly the lower conductors 11. The image pixels can also be separated from each other by surrounding them with ground electrodes, in order to improve the resolution and minimize interference. Depending on the processing technology used, active components, such as amplifiers, can also be placed in the image-pixel matrix. A thin wear-resistant layer can be formed on top of the upper conductors.

The conductors 10 and 11 can be implementedas metallic conductors and also as structures doped to become conductive in the circuit structure.

The advantage of frequency multiplexing is that the measuring of all the pixels takes place simultaneously, which shortens the measurement time and improves the resolution.

Comment: the simultaneous measuring of all the M×N pixels requires M×N mixers, which appears to be unrealistic already with even relatively small matrices. However, mixing can also be performed entirely digitally, in which case mixers will not be required, though the band requirement of the AD conversion will increase considerably.

In frequency multiplexing, the location of a pixel is coded to the value of the measurement frequency fn. The measurement interval is selected relative to the signals' frequencies, in such a way that the signals orthogonal to each other are given integrated over the measurement interval. This type of measurement procedure is used in known radio-communication technology, for example, in OFDM (Orthogonal Frequency Division Multiplexing) modulation. FIG. 2 shows schematically how the results of the impedance measurement of the second vertical pixel line P12, P22, P32, P42, and P52 can be separated from the output signal of the current amplifier 12 connected to the pixel line, by multiplying the signal with the aid of the mixers 13 at the frequency f1-f5 of each horizontal row. Problems in frequency measurement can be the linearity of the amplifiers 12 and the mixers 13, and the tolerance of the common-mode voltage.

Time multiplexing, on the other hand, takes place in a corresponding circuit one horizontal row 10 at a time, when values of the pixels of each row 11 (e.g., P12 in row 1) are read from the outputs of the amplifiers 12 and are saved in memory.

According to FIG. 3, the lower electrodes 11 are formed on top of the substrate 25 and the insulator layer 20 on top of this layer. The substrate 25 can be a normal circuit-board substrate or, for example, a plastic substrate. The technique according to the invention thus permits other techniques too than a silicon substrate to be used. Naturally, a silicon substrate is possible, but it is not the most advantageous alternative in terms of total economy, from the point of view of the invention. In terms of manufacturing technology, the most preferable solution is indeed obtained form a combination, in which the measurement matrix 10, 11 is formed on a circuit-board or plastic substrate and the electronics are implemented using a normal silicon-based technique.

For its part, the insulator layer 20 is either a conventional insulator layer, in which case the electrical effects of the protrusions 24 of the finger 23 can be measured at the intersections points of the electrodes 10 and 11 in the manner described above. Alternatively, the insulator layer 20 can be of a pyroelectrical material, the charge of which reacts to heat. The change in charge affects the measured capacitance.

FIG. 4 shows an alternative construction, in which a pyroelectrical layer 21 is located on top of the upper electrode 10.

FIG. 5 shows a solution, in which the lower electrode 11 is widened at the intersections of the electrodes 10 and 11, in order to increase the sensitivity of the measurement device.

Naturally, the locations of the electrodes can differ from the alternatives shown in the figures, so that the horizontal as well as the vertical electrodes can act as the upper electrodes. The same applies to the lower electrodes. The upper electrodes are preferably protected with a protective layer, in order to prevent mechanical and chemical wear.

According to the invention, the electrodes 10 and 11 need not be at right angles to each other, though in some situations a right-angled placing of the electrodes may be an advantageous solution, for instance for manufacturing-technology reasons.

The determining of ethanol content according to the invention takes place as follows.

It is known that when alcohol is drunk it is absorbed from the stomach into the blood circulation, which transports it to all parts of the body, to the cells, to the cell fluids, and also to the cells in the fingertips. The alcohol content of the cells in the fingertips is, after the absorption time from the drinking of the alcohol, of the same order as that of the blood. It is also known that the permittivity (dielectric constant) of alcohol is not constant, but depends on frequency, as is shown in FIG. 6. Thus, FIG. 6 shows the relative permittivity ∈ᵣ′ of alcohol (ethanol) and the loss term ∈ᵣ′ as a function of frequency. At a frequency of less than 100 GHz, the relative permittivity of alcohol is about 24 and decreases at higher frequencies, being about 14 at a frequency of 1 GHz and about 5 at a frequency of 10 GHz. A dependency of this kind is specific to ethyl alcohol. The alcohol content of the fingertip can be determined using a capacitive measurement principle of the following type: the finger is held on top of an electrode matrix. By means of the electrodes, the electronics connected to the matrix generate electrical fields in the fingertip at several different frequencies. When using only two different frequencies, suitable frequencies are in the order of 100 MHz and in the order of 2 GHz. The impedance between the electrodes is measured by means of the reading electronics connected to the electrode. The real part of the impedance depends on the loss term of the permittivity and the imaginary part on the permittivity. By calculating from the imaginary part the relation of the capacitances between the electrodes at these two frequencies, the alcohol content of the cell fluids of the fingertip can be determined precisely. If more frequencies are used, for example, 0.1 GHz, 0.4 GHz, 1 GHz, 2 GHz, and 4 GHz, some part combination of them, the curves shown in FIG. 6 can be applied to the measurement results and the measurement accuracy will improve considerably. Fingerprint sensors operate on essentially the same capacitive measurement principle. However, in these only one measurement frequency is used.

Thus, the method is based on the detection of the frequency dependence of the permittivity specific to ethanol. The relative permittivity of pure ethanol changes as a function of frequency, according to FIG. 6.

In the permittivity measurement of a water solution of ethanol, the permittivity of the solution can be assumed to follow approximately the equation

∈=α∈_{E}+b∈_{W}+c (1),

in which ∈ is the effective relative permittivity, ∈_{E} and ∈_{w} are the relative permittivities of ethanol and water, and a, b, and c are constants dependent on the volume fraction of ethanol, the temperature, and the measurement geometry.

The permittivity of a water solution of ethanol is measured at several different volume fractions of ethanol in the frequency range 200 MHz-6 GHz. FIG. 7 shows the adaption coefficients a, b, and c (ETAX coeff, H₂O coeff, and Level coeff) as a function of the volume fraction of ethanol, according to equation 1, adapted to the measurement results of the permittivity of a water solution of ethanol. It will be noticed that the adaption coefficient b of ethanol depends monotonously and relatively strongly on the volume fraction of ethanol in the solution. At low ethanol concentrations, the dependence is non-linear.

The measurements described above were repeated with an ethanol water solution, in which 2.3 g/l of table salt had been dissolved. This concentration of NaCl corresponds approximately to the salt concentration of human perspiration. FIG. 8 shows the adaption coefficients a, b, and c (ETAX coeff, H2O coeff, and Level coeff) according to equation 1 adapted to the measurement results of the permittivity of the ethanol and table-salt water solution, as a function of the volume fraction of ethanol. The adaption coefficient b of ethanol increases with the ethanol concentration, but a small degree of non-monotonicity can be seen in places. This is due to noise in the measurements. The coefficient c decreases monotonously as the ethanol concentration increases.

The permittivity adaption coefficient a of ethanol and the constant coefficient c depend on the ethanol concentration in the solution. By measuring permittivity at several different frequencies and by making a two parameter adaption to the measurement results, the ethanol concentration of the solution can be determined.

The subject of the invention thus has two basic ideas, the first of which is a touch-operated alcoholmeter, in which a capacitive alcoholmeter measures alcohol content by a touch contact of a fingertip, without drawing blood. The second idea is to combine a capacitive alcoholmeter and a fingerprint sensor. In such a device, there are a common sensor element and reading electronics, thus obtaining a significant cost benefit. The device can be used, for instance, in conjunction with vehicles as a personal identifier and as a power and alcohol lock, according to FIGS. 9 and 10. FIG. 9 shows a capacitive alcohol meter integrated in a car's steering wheel, which measures alcohol content from the palm, when it is touched. Alternatively, according to FIG. 10, the car's power lock can be replaced with a combination sensor according to the invention, a capacitive fingerprint and alcohol sensor, which identifies the driver and also acts as an alcohol lock. Naturally, such an alcohol lock/sensor according to FIG. 10 can operate in parallel with a normal power lock, in such a way that the power lock activates only when the person has been identified and alcohol has not been detected.

The invention can be applied in other capacitive measurement environments than the solutions described above.

In the present application, the term alcohol refers, in the preferred embodiment of the invention, to ethyl alcohol, ethanol.

In the present application, the term capacitive measurement refers to alternating electricity measurement, in which an electro-technically principally capacitive connection is formed between a person's skin and the measurement electrodes.

1. A method for measuring the ethanol content in a person, wherein
the ethanol content is measured from the surface of the person's skin, by measuring the skin's impedance using alternating electricity measurement with the aid of measurement electrodes.

2. The method according to claim 1, wherein the ethanol content is measured from the skin of the person's hand with the aid of at least two electrodes at several different frequencies.

3. The method according to claim 1, wherein the ethanol content is measured in such a way that the frequencies used are in the frequency range 0.1-4 GHz.

4. The method according to any of the above claim 1, wherein the electrodes are formed into an electrode matrix.

5. The method according to claim 1, wherein the electrodes are formed into a conductor matrix.

6. The method according to claim 1, wherein reading electronics connected to the electrodes are used to measure the impedance between the electrodes, to separate from the impedance the real part dependent on the loss term of the permittivity and the imaginary part dependent on the permittivity, and to calculate from the imaginary part the relation of the capacitances between the electrodes, from the results of at least two measurement frequencies, in order to determine the ethanol content of the skin.

7. The method according to claim 1, wherein, in the method
the effect of the capacitive surface form on the impedance between the electrode pair is measured,
an electrical model of the surface form is created from the measured impedances,
the electrodes pairs are created with the aid of a conductor matrix, which is formed of conductors formed into two layers insulated electrically from each other, at the intersections of which each electrode pair is formed, and
the impedance between each electrode pair is measured with the aid of either time or frequency multiplexing.

8. The method according to claim 7, wherein, in frequency multiplexing, the measurement interval is selected in such a way that the signals are orthogonal to each other, integrated over the given measurement period.

9. The method according to claim 7, wherein the conductor matrix is formed to be orthogonal.

10. The method according to claim 7, wherein, in the vicinity of the electrodes, a pyroelectrical material is used for detecting a surface form on the basis of heat.

11. The method according to claim 5, wherein the conductor matrix is formed on a circuit-board or plastic substrate.

12. The method according to claim 1, wherein the ethanol content is determined from the skin impedance by exploiting the equation
∈=α∈_{E}+b∈_{W}+c (2),
in which ∈ is the effective relative permittivity, ∈_{E} and ∈_{W} are the relative permittivities of ethanol and water, and a, b, and c are constants dependent on the volume fraction of ethanol, the temperature, and the measurement geometry.

13. An apparatus for measuring the ethanol content of a person,
wherein
the measurement means comprise electrodes, with the aid of which the impedance of the person's skin can be measured using an alternating-electricity circuit,
means for creating at least two measurement frequencies, and
means for determining the impedance of the skin at least the said two frequencies.

14. The apparatus according to claim 13, further comprising at least two electrodes, as well as means for conducting a measurement signal at several different frequencies to the electrodes.

15. The apparatus according to claim 13, further comprising means for creating measurement signals, the frequencies of which are in the range 0.1-4 GHz.

16. The apparatus according to claim 13, wherein the electrodes are formed into an electrode matrix.

17. The apparatus according to claim 13, wherein the electrodes are formed into a conductor matrix.

18. The apparatus according to claim 13, further comprising reading electronics connected to the electrodes, by means of which the impedance between the electrodes can be measured, the real part dependent on the loss term of the permittivity and the imaginary part dependent on the permittivity can be separated from the impedance, and the relation of the capacitances between the electrodes can be calculated from the imaginary part, in order to determine the skin ethanol content from the results of at least two measurement frequencies.

19. The apparatus for detecting ethanol according to claim 13, further comprising
means for measuring the effect of the surface form on the impedance between the electrode pair, and
means for creating an electrical model with the aid of the impedances measured from the surface form,
the electrode pairs are formed with the aid of a conductor matrix, which is formed of conductors formed into two layers insulated from each other electrically, at the intersections (P12, P22, P32, P42, P52) of which each electrode pair is formed, and
the apparatus comprises means for measuring the impedance between each electrode pair, with the aid of either time or frequency multiplexing.

20. The apparatus according to claim 19, further comprising, in frequency multiplexing, means for selecting the measurement interval, in such a way that the signals are orthogonal to each other integrated over the given measurement period.

21. The apparatus according to claim 19, wherein the conductor matrix is formed to be orthogonal.

22. The apparatus according to claim 19, wherein, in the vicinity of the electrodes, there is a pyroelectrical layer, for detecting a surface form on the basis of heat.

23. The method according to claim 5, wherein the conductor matrix is formed on a circuit-board or plastic substrate.

24. A method for detecting a surface form, such as a fingerprint, in which method
the effect of the surface form on the impedance between an electrode pair is measured capacitively, and
an electrical model of the surface form is created from the measured impedances, wherein
the electrode pairs are created with the aid of a conductor matrix, which is formed of conductors formed into two layers insulated from each other electrically, at the intersections (P12, P22, P32, P42, P52) of which each electrode pair is formed, and
the impedance between each electrode pair is measured with the aid of either time or frequency multiplexing.

25. An apparatus for detecting a surface form, such as a fingerprint, which apparatus comprises
means for measuring the effect of the surface form on the impedance between an electrode pair, and
means for creating an electrical model of the surface form, with the aid of the measured impedances,
wherein
the electrode pairs are formed with the aid of a conductor matrix, which is formed of conductors formed into two layers insulated electrically from each other, at the intersections (P12, P22, P32, P42, P52) of which each electrode pair is formed, and
the apparatus comprises means for measuring the impedance between each electrode pair with the aid of either time or frequency multiplexing.
